(19) United States
(12) Patent Application Publication
(10) Pub. No.: US 2003/0163030 A1
(43) Pub. Date: Aug. 28, 2003

(54) Hollow endoscopy

(76) Inventor: Moises A. Arriaga, Pittsburgh, PA (US)

Correspondence Address: Barbara E. Johnson, Webb Ziesenheim Logsdon Orkin & Hanson, P.C., 700 Koppers Building, 436 Seventh Avenue, Pittsburgh, PA, 15219, US

(21) Appl. No.: 10/371,036

(22) Filed: Feb. 20, 2003

Related U.S. Application Data
(60) Provisional application No. 60/359,577, filed on Feb. 25, 2002.

(51) Int. Cl. A61B 1/06

(52) U.S. Cl. 600/182

(57) ABSTRACT

An endoscope includes a body defining a bore that extends therethrough. A number of fiber optic cables extend at least partially through the body and terminate adjacent one end of the bore. The number of fiber optic cables includes a first subset having their ends opposite the body coupled to a light source. The number of fiber optic cables can also include one or more additional subsets having their ends opposite the body coupled to a viewer.

### CROSS-REFERENCE TO RELATED APPLICATION

The present application claims priority from U.S. Provisional Patent Application Serial No. 60/359,577, filed Feb. 25, 2002.

### BACKGROUND OF THE INVENTION

1. Field of the Invention

The present invention relates to an apparatus to facilitate viewing of objects in confined spaces.

2. Description of Related Art

Advances in endoscopy have been critical to developing minimally invasive surgery in a broad array of surgical disciplines including general surgery, gynecology, urology, plastic surgery, laryngology and rhinology. The basic principles involve illumination through specially designed lighted rigid tubes which encompass a high quality optic system.

These endoscopes permit visualization of instrumentation disposed adjacent the endoscope. In abdominal surgery, the surgeon has the breadth of the abdominal cavity in which to manipulate an instrument. Even in narrower spaces, such as the paranasal sinuses, surgeons are able to manage aggressive disease with minimal external incisions—a sharp contrast to previous techniques.

Ear surgery has not benefited as significantly as other disciplines. Although endoscopes have been proposed as adjuncts to routine open microscopic approaches, otologic endoscopy has not replaced standard binocular microscopy as the illumination/visualization technique of choice for most ear surgical procedures. Specific aspects of ear surgery that have delayed the evolution of endoscopic otologic techniques include:

1. Size: Anatomically, the spaces of the ear are small and rigidly confined. The ear canal varies in diameter from 4-8 mm on average. Thus, even a narrow diameter endoscope occupies a significant percentage of the lumen and obstructs passage of instruments adjacent the endoscope;

2. Stability: No standard system exists for supporting the endoscope in the ear canal or mastoid to permit bimanual instrumentation during endoscopic viewing of the ear. Ear speculum holders are not designed to support the weight of an endoscope and slippage is common. Holding the endoscope with one hand and manipulating an instrument with the other, such as commonly done in sinus endoscopy, is not practical because of the microscopic structures under consideration. Bimanual, stable visualization and instrumentation is critical for surgery of the ossicular chain;

3. Visualization: As surgeons have tried smaller endoscopes, the problem of visualization develops. First, image quality erodes if the endoscope is too small. But the main problem is that the surgeon can block the view of the surgical field by placing objects directly in front of the endoscope's field of view.

Despite the apparent limitations, endoscopes in ear surgery offer the following advantages over binocular microscopy:

1. Minimal invasion: To accomplish adequate exposure for microscopic ear surgery, otologic surgeons often make postauricular incisions and reshape the bone of the ear canal. Although such steps facilitate precision and desirable surgical results, they do add additional invasion and requisite discomfort and healing requirements for the patient. Ideally, endoscopes could limit the need for additional surgical wounds to accomplish adequate exposure.

2. Illumination: During standard microscopic ear surgery, the light from the microscope illuminates past the surgeon's hands and instruments. Then the light must reflect back to the lens, again past the surgeon's instruments and hands. This arrangement causes occasional obstruction of the surgical field and may require awkward rearrangement of the surgeon's hands.

It is, therefore, desirable to provide an endoscope that facilitates illumination, viewing and manipulation of instruments in a field of view of the endoscope with minimal interference between the illumination, the acquisition of a view and the instruments. Still other desirable features of the present invention will become apparent to those of ordinary skill in the art upon reading and understanding the following detailed description.

### SUMMARY OF THE INVENTION

The invention is an endoscope that includes a body defining a bore that extends therethrough. A plurality of fiber optic cables extends at least partially through the body and terminates adjacent one end of the bore. The plurality of fiber optic cables includes a first subset of fiber optic cables having their ends opposite the body coupled to a source of light. As used herein, the term “subset” means a set consisting of elements of a given set that can be the same as the given set or smaller.

The bore can include therein a restriction configured to prevent the passage of the barrel of a telescope therethrough. The bore can have the shape of a truncated cone or a cylinder.

The plurality of fiber optic cables can further include a second subset of fiber optic cables having their ends opposite the body coupled to a viewer. The viewer can be operative for displaying an image received by the second subset of fiber optic cables as a monocular image. The viewer can be one of a video monitor or an eye piece.

The first and second subsets of fiber optic cables can terminate in concentric rings adjacent the one end of the bore.

The plurality of fiber optic cables can further include a third subset of fiber optic cables coupled to the viewer. The second and third subset of fiber optic cables can terminate in a manner to receive different perspective views from a field of view. The viewer can be operative for displaying an image received from the field of view by each of the second and third subsets of fiber optic cables separately, e.g., as a stereoscopic image.

The first subset of fiber optic cables can terminate adjacent the one end of the bore in a first ring and the second and third subsets of fiber optic cables can terminate adjacent the one end of the bore in a second ring that is concentric with the first ring.

The second and third subsets of fiber optic cables can terminate adjacent the one end of the bore on opposite sides thereof or in an alternating manner around the one end of the bore.

The invention is also a hollow speculum endoscope comprising a generally frustum shaped body having an apex and a base at opposite ends thereof and a cavity that extends between the apex and the base. A first light transmitter extends at least partially through the body. The first light transmitter has a terminal end which is encased in the body adjacent the apex thereof. The first light transmitter has an origin end configured to be coupled to a source of light which is operative to transmit light through said first light transmitter for projection from the terminal end thereof.

The cavity can include a restriction therein configured to avoid the passage of the barrel of a telescope therethrough. The cavity can be configured to receive one or more surgical instruments therethrough.

The endoscope can also include a second light transmitter having an origin end which is encased in the body adjacent the apex thereof. The second light transmitter extends from the origin end thereof and at least partially through the body. The second light transmitter has a terminal end configured to be coupled to a viewer which is operative for enlarging an image acquired by the second light transmitter from a field of view which is disposed on a side of the apex of the body opposite the base of the body.

The first and second light transmitters can define concentric rings adjacent the apex of the body. The viewer can be operative for displaying the image received by the second light transmitter as a monocular image.

The second light transmitter can include first and second subsets of light transmitting elements coupled to provide to the viewer first and second perspectives of the field of view. The viewer can be operative for displaying the perspectives of an object positioned in the field of view received from the first and second subsets of light transmitting elements as a stereoscopic image of the object. The first and second subsets of light transmitting elements can terminate on opposite sides of the cavity adjacent the apex of the body or in an alternating manner around the end of the cavity adjacent the apex of the body.

### BRIEF DESCRIPTION OF THE DRAWINGS

FIG. 1a is a perspective view of an endoscope including a first subset of light transmitters coupled to a light source;

FIG. 1b is a view taken along lines Ib-Ib in FIG. 1a;

FIG. 2 is a view from the apex end of an endoscope having an egg shaped body;

FIG. 3a is a perspective view of the endoscope shown in FIG. 1a further including a second plurality of light transmitters coupled to a viewer;

FIG. 3b is a view taken along lines IIIb-IIIb in FIG. 2a;

FIG. 4 is a perspective view of the endoscope shown in FIG. 1a further including second and third pluralities of light transmitters disposed on opposite sides of the endoscope and separately coupled to a viewer;

FIG. 5 is a perspective view of the endoscope shown in FIG. 4 wherein different sets of light transmitters comprise the second and third plurality of light transmitters that are coupled to the viewer; and

FIG. 6 is a cross-sectional perspective view of an endoscope including a bore having a restriction therein for contacting an embossment, or other such structure, adjacent the image receiving end of a telescope.

### DETAILED DESCRIPTION OF THE INVENTION

The present invention will be described with reference to the accompanying figures where like reference numbers correspond to like elements.

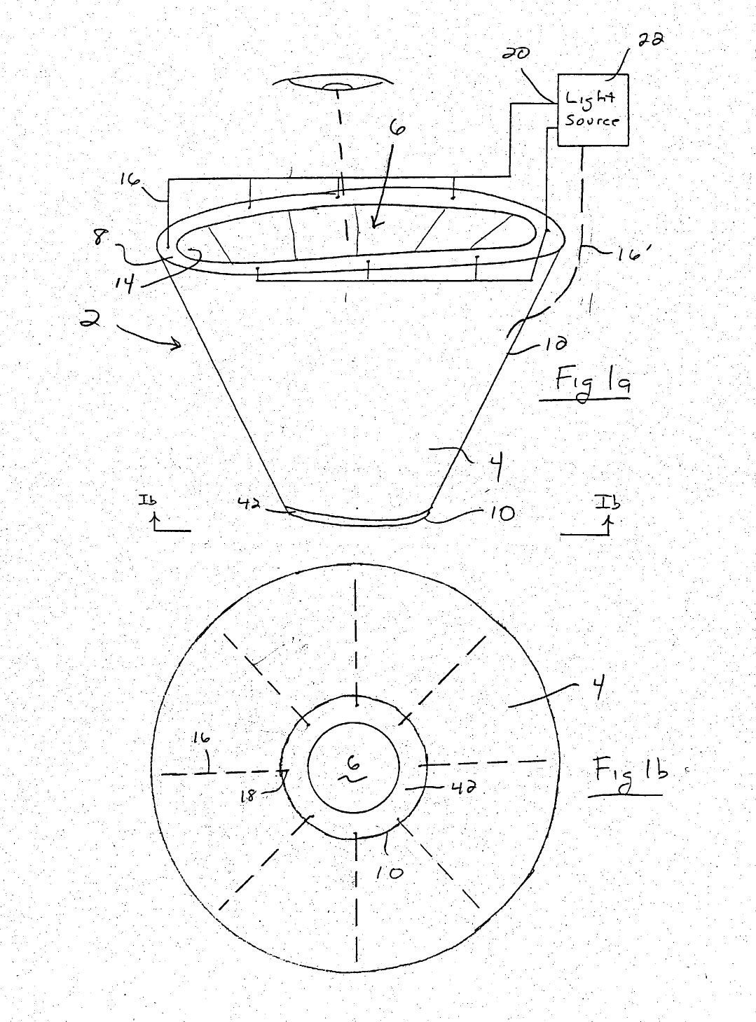

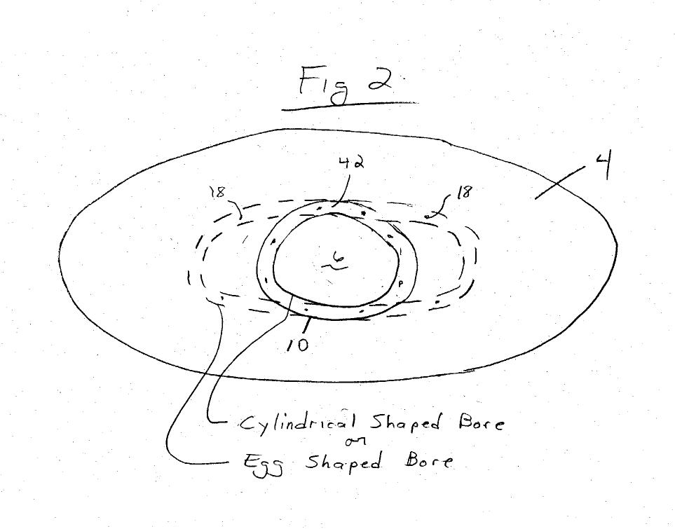

With reference to FIGS. 1a and 1b, a hollow speculum endoscope 2 includes a body 4 having a bore 6 that extends between a first end 8 of body 4 and a second end 10 of body 4. Body 4 and bore 6 can have a generally frustum shape wherein first end 8 defines a base and second end 10 defines an apex of body 4. However, the description of body 4 and bore 6 as having frustum shapes is not to be construed as limiting the invention, since body 4 and/or bore 6 can have any suitable shape. For example, body 4 and bore 6 can have cylindrical shapes; body 4 can have a frustum shape while bore 6 can have a cylindrical shape; or, as shown in FIG. 2, body 4 can have an ovoid or egg shape and bore 6 can have an ovoid or cylindrical shape.

Body 4 is defined by an outer wall 12 and an inner wall 14 positioned in spaced relation between first end 8 and second end 10. Inner wall 14 also defines bore 6.

Between outer wall 12 and inner wall 14, body 4 can be solid, hollow or some combination of solid and hollow between first end 8 and second end 10. One or more light transmitters 16, such as a fiber optic cable, can be positioned in body 4 between outer wall 12 and inner wall 14. Each light transmitter 16 has a terminal or distal end 18 that terminates adjacent second end 10. An origin or proximal end 20 of each light transmitter 16 can be optically coupled to a light source 22 which is operative to provide light to each light transmitter 16 having its proximal end 20 coupled thereto. Desirably, the proximal end 20 of each light transmitter 16 is coupled directly to light source 22.

Each light transmitter 16 conveys light received from light source 22 to second end 10 of body 4 whereupon said light exits light transmitter 16 and travels toward a field of view disposed on a side of second end 10 opposite first end 8.

Each object disposed in the field of view is illuminated by light exiting the distal end of light transmitter 16. For example, when second end 10 of body 4 is positioned in the ear canal of a patient, light exiting the distal end of each light transmitter illuminates ear structure disposed in the field of view. Once illuminated, each object in the field of view can be observed simply by peering, either with the naked eye or via a telescope, through bore 6 adjacent first end 8 of body 4. If necessary, one or more instruments (not shown) can be projected through bore 6 into operative relation with the one or more objects in the field of view. More specifically, the distal end of each instrument can be projected through bore 6 while the proximal end of each instrument can remain outside of bore 6 on a side of first end 8 opposite second end 10 for manipulation by a user to effect a desired procedure on an object in the field of view. Once the desired procedure is complete, the distal end of each instrument can be withdrawn from the field of view via bore 6.

In FIG. 1a, each light transmitter 16 enters body 4 at first end 8 thereof. However, if desired, each light transmitter 16' (shown in phantom) can enter body 4 via outer wall 12 or inner wall 14 intermediate first end 8 and second end 10. Accordingly, the illustrated entry point of each light transmitter 16 into body 4 is not to be construed as limiting the invention.

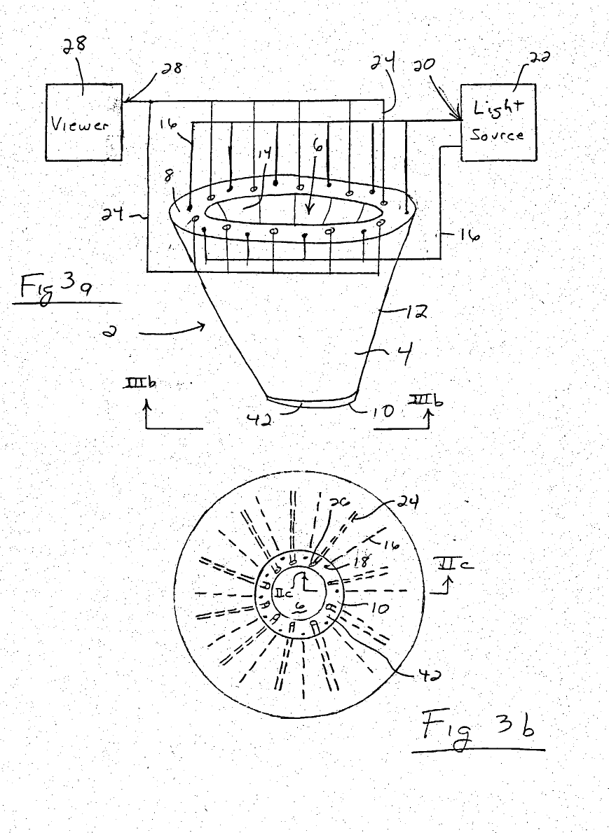

With reference to FIGS. 3a-3b, and with continuing reference to FIGS. 1a and 1b, hollow speculum endoscope 2 can also include one or more light transmitters 24, such as a fiber optic cable, which extend between first end 8 and second end 10 of body 4 in the manner described above for each light transmitter 16. Each light transmitter 24 has an origin or proximal end 28 which initiates adjacent second end 10 of body 4. A terminal or distal end of each second light transmitter 24 is coupled to a viewer 30 which, in FIG. 3a, is operative to display a monocular image of one or more objects in the field of view of endoscope 2. Viewer 30 can be any conventional device that enables a user to visualize objects positioned in the field of view of endoscope 2. Examples of viewer 30 include a goggle or a video display.

In use, light source 22 and light transmitters 16 co-act to project light onto one or more objects positioned in the field of view and light transmitters 24 and viewer 30 co-act to transfer light reflected from said objects to viewer 30 for visualization by a user. As with the embodiment of endoscope 2 shown in FIG. 1a, the distal end of one or more instruments, such as medical instruments, can be inserted through bore 6 of endoscope 2 shown in FIG. 3a for manipulation in the field of view of endoscope 2. Hence, when used in connection with an instrument, endoscope 2 shown in FIG. 3a enables light to be provided to the field of view and enables images to be acquired from the field of view while, at the same time, providing unobstructed entry of one or more instruments into the field of view for manipulation during a procedure. In addition, bore 6 of endoscope 2 shown in FIG. 3a can be utilized to facilitate viewing of one or more objects in the field of view in the manner discussed above in connection with the embodiment of endoscope 2 shown in FIG. 1a.

The distal ends 18 and proximal ends 26 of light transmitters 16 and 24, respectively, can be arranged in any desirable configuration. One such configuration, shown in FIG. 3b, includes the distal ends of light transmitters 8 and the proximal ends of light transmitters 24 arranged in concentric circles. However, this is not to be construed as limiting the invention since the distal ends of light transmitters 18 and the proximal ends 26 of light transmitters 24 can be arranged in any desirable manner.

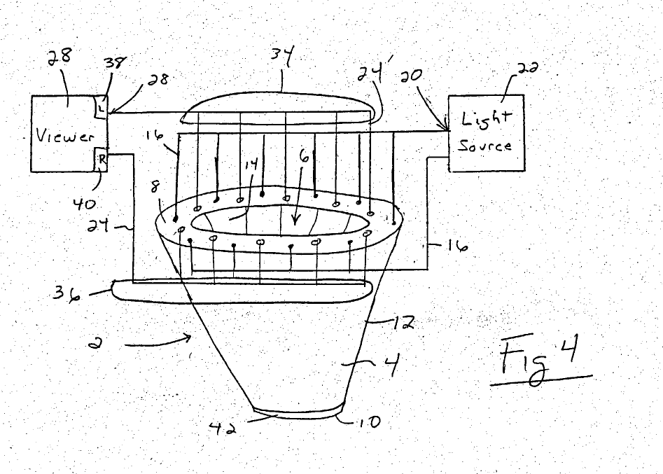

With reference to FIG. 4, and with continuing reference to FIGS. 1a, 1b, 3a and 3b, light transmitters 24 can be split into two groups 34 and 36. As shown in FIG. 4, each light transmitter 24 of group 34 is disposed on one side of body 4 while each light transmitter 24 of group 36 is disposed on the other side of body 4. Light transmitters 24 of groups 34 and 36 can be coupled to separate inputs 38 and 40, respectively, of viewer 30. Because light transmitters 24 of groups 34 and 36 are disposed on opposite sides of body 4, they can provide first and second perspectives of the field of view to viewer 30 which can be operative for displaying the first and second perspectives as a stereoscopic image.

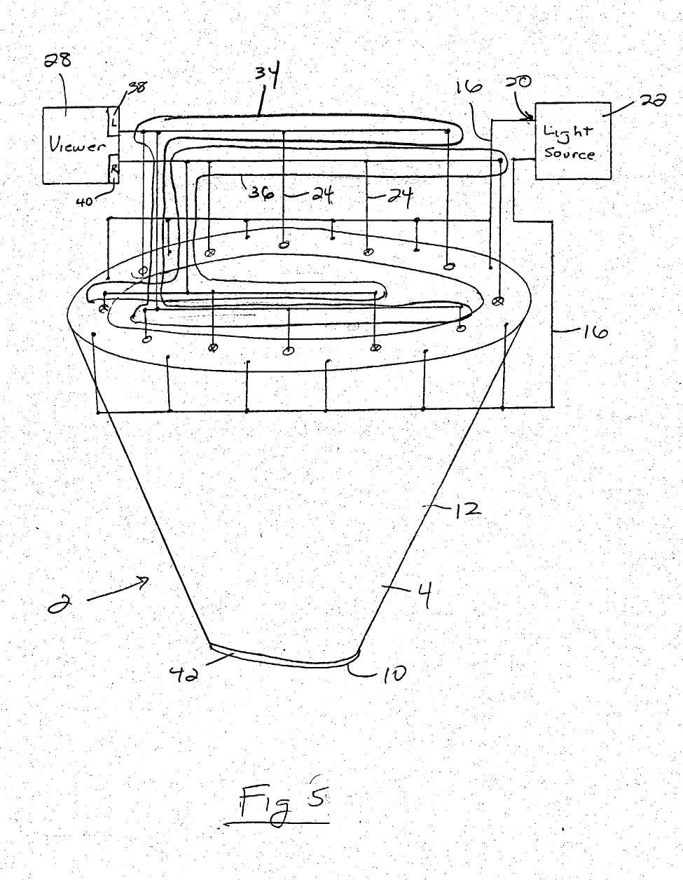

With reference to FIG. 5, and with continuing reference to FIG. 4, instead of light transmitters 24 of groups 34 and 36 being disposed on opposite sides of body 4, light transmitters 24 of groups 34 and 36 can be coupled in an alternating manner to inputs 38 and 40, respectively, of viewer 30 for stereoscopic viewing of objects in the field of view. Other than the coupling of light transmitters 24 of groups 34 and 36 in an alternating manner to the inputs 38 and 40 of viewer 30, the embodiment of endoscope 2 shown in FIG. 5 is the same as the embodiment of endoscope 2 shown in FIG. 4.

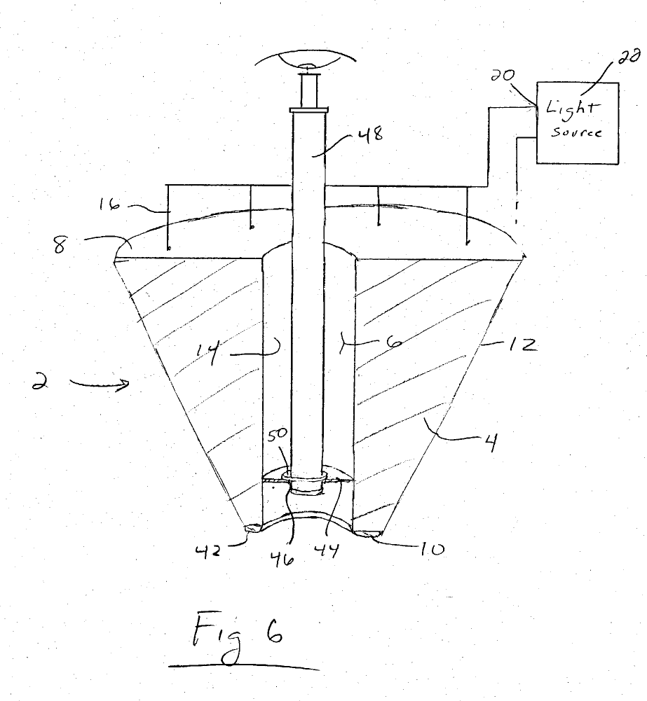

With reference to FIG. 6, the embodiment of endoscope 2 shown in FIG. 6 is similar to the embodiment of endoscope 2 shown in FIG. 1 except in FIG. 6 bore 6 has a cylindrical shape and bore 6 includes a restriction 44 therein. The description of restriction 44 as part of cylindrical bore 6 in FIG. 6, however, is not to be construed as limiting the invention. Restriction 44 can include an aperture 46 configured to receive the barrel of a telescope 48 therethrough. The barrel of telescope 48 can include an embossment 50 positioned at a suitable location thereon to avoid telescope 48 from passing through bore 6. In use, the image capturing end of telescope 48 is inserted via first end 8 into barrel 6 and into aperture 46 of restriction 44 until embossment 50 contacts a surface of restriction 44. Thereafter, a user can peer into the viewing end of telescope 48 to observe objects positioned in the field of view of endoscope 2. When a procedure is complete, telescope 48 can be withdrawn from bore 6.

In FIG. 6, restriction 44 is shown as having a washer shape. However, this is not to be construed as limiting the invention since any form of restriction that avoids movement of the image receiving end of telescope 48 past a desired position in bore 6 is envisioned.

As can be seen, the present invention is a hollow speculum endoscope that enables light and instruments to be provided to a field of view with minimal interference therebetween. The present invention also enables objects in the field of view to be visualized, either directly, e.g., with the naked eye or via telescope 48, or indirectly, e.g., via viewer 30, with minimal interference.

The invention has been described with reference to the preferred embodiments. Obvious modifications and alterations will occur to others upon reading and understanding the preceding detailed description. For example, second end 10 can include a clear bezel 42 that enables light from light transmitters 16 to pass therethrough while enabling light reflected from objects in the field of view to pass therethrough into light transmitters 24. Bezel 42 avoids fluid and particulate matter from contacting the ends of light transmitters 16 and 24 disposed adjacent second end 10. It is intended that the invention be construed as including all such modifications and alterations insofar as they come within the scope of the appended claims or the equivalents thereof.

The invention claimed is:

1. An endoscope comprising:
a body defining a bore that extends therethrough; and
a plurality of fiber optic cables extending at least partially through the body and terminating adjacent one end of the bore, the plurality of fiber optic cables including a first subset of fiber optic cables having their ends opposite the body coupled to a source of light.

2. The endoscope of claim 1, wherein the bore includes therein a restriction configured to avoid passage of a barrel of a telescope therethrough.

3. The endoscope of claim 1, wherein the bore has the shape of one of a (i) truncated cone and (ii) a cylinder; and (iii) an ovoid.

4. The endoscope of claim 1, wherein the plurality of fiber optic cables further includes a second subset of fiber optic cables having their ends opposite the body coupled to a viewer.

5. The endoscope of claim 4, wherein the viewer is operative for displaying the image received by the second subset of fiber optic cables as a monocular image.

6. The endoscope of claim 4, wherein the viewer is one of a video monitor and an eyepiece.

7. The endoscope of claim 4, wherein the first and second subsets of fiber optic cables terminate in concentric rings adjacent the one end of the bore.

8. The endoscope of claim 4, wherein:
the plurality of fiber optic cables further includes a third subset of fiber optic cables coupled to the viewer;
the second and third subset of fiber optic cables terminate in a manner to receive different perspective views from a field of view; and
the viewer is operative for displaying an image received from the field of view by each of the second and third subsets of fiber optic cables separately.

9. The endoscope of claim 8, wherein the viewer is operative for displaying the image received by the second and third subsets of fiber optic cables as a stereoscopic image.

10. The endoscope of claim 8, wherein:
the first subset of fiber optic cables terminates adjacent the one end of the bore in a first ring; and
the second and third subsets of fiber optic cables terminate adjacent the one end of the bore in a second ring that is concentric with the first ring.

11. The endoscope of claim 8, wherein:
the second and third subsets of fiber optic cables terminate adjacent the one end of the bore; and
the second and third subsets of fiber optic cables terminate on opposite sides of the one end of the bore.

12. The endoscope of claim 8, wherein:
the second and third subsets of fiber optic cables terminate adjacent the one end of the bore; and
the fiber optic cables of the second and third subsets of fiber optic cables terminate in an alternating manner around the one end of the bore.

13. A hollow speculum endoscope comprising:
a body having an apex and a base at opposite ends thereof and a cavity that extends between the apex and the base; and
a first light transmitter extending at least partially through the body, said first light transmitter having a terminal end which is received in the body adjacent the apex of the body, said first light transmitter having an origin end configured to be coupled to a source of light which is operative to transmit light through said first light transmitter for projection from the terminal end thereof.

14. The endoscope of claim 12, wherein the cavity is configured to receive one or more surgical instruments therethrough.

15. The endoscope of claim 12, wherein the cavity includes a restriction configured to avoid passage of a barrel of a telescope therethrough.

16. The endoscope of claim 12, further including:
a second light transmitter having an origin end which is received in the body adjacent the apex of the body, said second light transmitter extending from the origin end thereof and at least partially through the body, said second light transmitter having a terminal end configured to be coupled to a viewer which is operative for enlarging an image acquired by the second light transmitter from a field of view which is disposed on a side of the apex of the body opposite the base of the body.

17. The endoscope of claim 16, wherein the first and second light transmitters define concentric rings adjacent the apex of the body.

18. The endoscope of claim 16, wherein the viewer is operative for displaying the image received by the second light transmitter as a monocular image.

19. The endoscope of claim 16, wherein the second light transmitter includes first and second subsets of light transmitting elements coupled to provide to the viewer first and second perspectives of the field of view.

20. The endoscope of claim 19, wherein the viewer is operative for displaying the perspectives of an object positioned in the field of view received from the first and second subsets of light transmitting elements as a stereoscopic image of the object.

21. The endoscope of claim 19, wherein the first and second subsets of light transmitting elements terminate in an alternating manner around the end of the cavity adjacent the apex of the body.
